(19) United States
(12) Patent Application Publication
(10) Pub. No.: US 2026/0248893 A1
(43) Pub. Date: Aug. 27, 2026

(54) CONTROLLED-RELEASE PREPARATION AND PREPARATION METHOD THEREFOR

(71) Applicant: ZHEJIANG UNIVERSITY, Zhejiang (CN)

(72) Inventors: Zhen GU, Zhejiang (CN); Jinqiang WANG, Zhejiang (CN); Jianchang XU, Zhejiang (CN)

(21) Appl. No.: 19/163,371

(22) PCT Filed: Mar. 15, 2024
(86) PCT No.: PCT/CN2024/081997

§ 371 Date: Sep. 8, 2025

(30) Foreign Application Priority Data
Mar. 17, 2023 (CN) 202310263461.7

(51) Int. Cl. A61K 38/28; A61K 9/50

(52) U.S. Cl. CPC A61K 38/28; A61K 9/5031; A61K 9/5052; A61K 9/5089

(57) ABSTRACT

A controlled-release preparation and a preparation method therefor. Specifically, the controlled-release preparation comprises a drug crystal and a polymer coating wrapping same. The method for preparing the controlled-release preparation comprises the following steps: subjecting a monomer to a polymerization reaction on the surface of the drug crystal to form a polymer coating, or subjecting a polymer to a cross-linking reaction on the surface of the drug crystal to form a polymer coating. The controlled-release preparation has high drug loading capacity and long drug release time, can achieve long-acting zero-level release of a drug, the preparation method is simple, and the encapsulation efficiency is high.

The present application claims the right of the priority of Chinese patent application 2023102634617 filed on Mar. 17, 2023. The content of the above Chinese patent application is incorporated herein by reference in its entirety.

### TECHNICAL FIELD

The present disclosure relates to the field of drug delivery, and particularly to a controlled-release preparation and a preparation method therefor.

### BACKGROUND

Chronic diseases, such as cardiovascular and cerebrovascular diseases, diabetes, and cancer, are a kind of disease with insidious onset, long course, persistent progression, and complex etiology. They are characterized by high incidence rates, low cure rates, high mortality rates, etc., and are accompanied by serious complications. The treatment of chronic diseases typically requires long-term, uninterrupted drug intake, significantly impacting patients' quality of life and imposing a heavy burden on families. Long-acting sustained-release drug preparations can slowly release drugs in the body, maintaining stable blood drug concentrations for a long time, making them one of the effective approaches for treating chronic diseases.

High drug loading is a necessary condition for constructing long-acting sustained-release drug preparations. In addition, drug carriers with high drug loading can reduce the frequency of drug intake, improve patient compliance, and reduce the use of materials/excipients and related side effects. However, due to compatibility issues between the drug carrier and the drug, the drug loading is usually limited, generally below 30 wt %. Therefore, significantly increasing the drug loading of drug carriers remains a challenge.

Traditional drug carriers with first-order release characteristics are difficult to effectively deliver certain drug molecules with narrow therapeutic windows and short half-lives. To address the above problems, zero-order release has emerged. This type of drug delivery system is capable of releasing the drug at a constant rate. If the clearance rate of drug molecules in vivo matches the release rate, zero-order drug release can maintain a stable blood drug concentration. Achieving zero-order release requires meeting the following conditions: 1. There is an excess of undissolved drug inside the drug carrier; 2. The drug dissolution rate is faster than the diffusion rate; 3. The drug diffusion rate remains relatively constant. Currently, most systems exhibiting zero-order release characteristics are implantable devices, such as osmotic pumps, intravaginal rings, microchips, and microneedles. Constructing an injectable drug carrier with zero-order release characteristics remains a significant challenge.

### SUMMARY

The technical problem to be solved by the present disclosure is to overcome the issues of existing drug carriers, such as low drug loading, short drug release time, and inability to achieve zero-order release, thereby providing a controlled-release preparation and a preparation method therefor. The controlled-release preparation of the present disclosure has an ultra-high drug loading and a long drug release time, enabling long-term zero-order release of drugs. Moreover, the preparation method is simple and exhibits high encapsulation efficiency.

The present disclosure addresses the above technical problems through the following technical solutions.

The present disclosure provides a controlled-release preparation, comprising a drug crystal and a polymer coating encapsulating the drug crystal.

The polymer is a polymer that can be formed by in situ polymerization of a monomer on the surface of the drug crystal, or the polymer is a polymer that can be adsorbed on the surface of the drug crystal and cross-linked, such as a dopa-modified polymer (i.e., a polymer containing a catechol structure), preferably polydopamine, poly-levodopa, or mussel adhesive protein. The polymer coating exists in the form of a polymer film.

The drug is any type of drug that can form a crystal, such as human insulin, an insulin analog, glucagon-like peptide-1 (GLP-1), a hyperglycemic hormone, or an analgesic, preferably recombinant human insulin, insulin glargine, insulin detemir, insulin aspart, insulin lispro, insulin glulisine, low-protamine zinc recombinant human insulin, semaglutide, glucagon, tetracaine, or bupivacaine.

The particle size of the drug crystal may be at the nanometer, micrometer, millimeter, or centimeter scale, depending on the specific drug crystal particle size, and may specifically range from 10 nm to 2 cm, preferably from 0.1 μm to 1000 μm, and more preferably from 5 μm to 56 μm.

In a preferred embodiment of the present disclosure, the drug is human insulin or an insulin analog (such as recombinant human insulin or insulin glargine), and the polymer is polydopamine or poly-levodopa.

In a preferred embodiment of the present disclosure, the drug is human insulin or an insulin analog (such as recombinant human insulin), and the polymer is mussel adhesive protein.

The mass ratio of the drug crystal to the polymer coating may be 1:(0.2-1.2), preferably 1:(0.2-0.8), and more preferably 1:0.4 or 1:0.2.

The thickness of the polymer coating may be 50-1000 nm, preferably 80-485 nm, and more preferably 161.54 nm.

The controlled-release preparation preferably consists of the drug crystal and the polymer coating encapsulating the drug crystal.

The controlled-release preparation may be any suitable dosage form for administration in the art, such as an injection dosage form (e.g., subcutaneous injection), an oral dosage form, or an implantable device.

The drug loading of the controlled-release preparation is preferably 50%-99%, and more preferably 60%-93%.

The encapsulation efficiency of the controlled-release preparation is preferably 60%-99.8%, and more preferably 73.4%-99.8%.

The present disclosure also provides a preparation method for the above controlled-release preparation, comprising the following step: subjecting a monomer to a polymerization reaction on the surface of a drug crystal to form a polymer coating.

The polymer is a polymer that can be formed by in situ polymerization of the monomer on the surface of the drug crystal, and the conditions of the polymerization reaction can be selected according to the polymerization conditions required for the polymer. The operation of the polymerization reaction may specifically be: mixing the monomer with the drug crystal in a solvent to carry out the polymerization reaction.

The drug crystal is selected as described above.

The monomer is selected according to the monomer required to form the polymer. The mass ratio of the drug to the monomer may be 1:(0.2-1.2), preferably 1:(0.2-0.8), and more preferably 1:0.4.

The solvent is a solvent that renders the drug crystal insoluble. For example, a buffer that renders the drug crystal insoluble may be selected.

The polymerization reaction may be carried out in the presence of a catalyst depending on polymerization requirements. When a catalyst is used, the preferred mixing sequence is to first mix the monomer and the drug crystal, and then mix with the catalyst.

The polymerization reaction is preferably carried out under stirring conditions.

The time and number of the polymerization reaction are selected based on the desired thickness of the polymer coating. When multiple polymerization reactions are required for multiple encapsulations, the specific operations are as follows: after a single polymerization reaction is completed, the drug crystal encapsulated with the resulting polymer coating is collected and then mixed with the monomer for another polymerization reaction until the desired thickness of the polymer coating is obtained.

The temperature of the polymerization reaction is selected according to the temperature required for polymerization without affecting the activity of the drug.

In a preferred embodiment of the present disclosure, the drug is insulin or an insulin analog (e.g., recombinant human insulin or insulin glargine).

In this preferred embodiment, the monomer is preferably dopamine or levodopa. The mass ratio of the drug crystal to the monomer may be 1:(0.2-1.2), preferably 1:(0.4-0.8), and more preferably 1:0.4. The mass concentration of the monomer in the solvent may be 0.5-3 mg/mL, preferably 1-2 mg/mL, and more preferably 1 mg/mL.

In this preferred embodiment, the solvent is a buffer; the pH value of the buffer may be 4-9, preferably 5-8, and more preferably 7. The buffer may be a disodium hydrogen phosphate-citrate buffer, Tris buffer, or PBS buffer. The concentration of the buffer may be 10-200 mM, preferably 10-182 mM, and more preferably 182 mM. Most preferably, the buffer is a disodium hydrogen phosphate-citrate buffer with a concentration of 182 mM and a pH of 7.

In this preferred embodiment, the polymerization reaction is carried out in the presence of an oxidizing agent. The oxidizing agent is preferably a persulfate (e.g., ammonium persulfate), a divalent copper salt, a trivalent iron salt, a permanganate (e.g., potassium permanganate), or a periodate (e.g., sodium periodate), and more preferably ammonium persulfate. The mass concentration of the oxidizing agent in the solvent is 0.3-1.2 mg/mL, preferably 0.4-0.8 mg/mL, and more preferably 0.6 mg/mL.

In this preferred embodiment, the time of the polymerization reaction is preferably 2-12 hours, more preferably 2-8 hours, and most preferably 4 hours.

In this preferred embodiment, the temperature of the polymerization reaction may be room temperature, for example, 4-37° C., preferably 20-30° C.

Conventionally, the preparation method may further comprise post-treatment steps, such as centrifugation, washing (e.g., with PBS), and drying (e.g., freeze-drying).

The present disclosure also provides a preparation method for the above controlled-release preparation, comprising the following step:

subjecting a polymer to a cross-linking reaction on the surface of the drug crystal to form a polymer coating.

The polymer is a polymer that can be adsorbed on the surface of the drug crystal and cross-linked, and the conditions of the cross-linking reaction can be selected according to the cross-linking conditions required by the polymer. The operation of the cross-linking reaction may specifically be: mixing the polymer with the drug crystal in a solvent to carry out the cross-linking reaction.

In the cross-linking reaction, the drug crystal is selected as described above.

In the cross-linking reaction, the solvent is a solvent that renders the drug crystal insoluble. For example, a buffer that renders the drug crystal insoluble may be selected.

The cross-linking reaction may be carried out in the presence of a catalyst as needed. In the cross-linking reaction, when a catalyst is used, the preferred mixing sequence is to first mix the polymer with the drug crystal, and then mix with the catalyst.

The cross-linking reaction is preferably carried out under stirring conditions.

The time and number of the cross-linking reaction are selected based on the desired thickness of the polymer coating. When multiple cross-linking reactions are required for multiple encapsulations, the specific operations are as follows: after a single cross-linking reaction is completed, the drug crystal encapsulated with the resulting polymer coating is collected and then mixed with the polymer for another cross-linking reaction until the desired thickness of the polymer coating is obtained.

The temperature of the cross-linking reaction is selected according to the temperature required for cross-linking without affecting the activity of the drug.

In a preferred embodiment of the present disclosure, the drug is insulin or an insulin analog (such as recombinant human insulin), and the polymer is mussel adhesive protein.

The mass ratio of the drug crystal to the polymer may be 1:(0.2-1.2), preferably 1:(0.2-0.4), and more preferably 1:0.2. The mass concentration of the polymer in the solvent may be 0.5-3 mg/mL, preferably 1-2 mg/mL, and more preferably 1 mg/mL.

In this preferred embodiment, the solvent is a buffer; the pH value of the buffer may be 4-9, preferably 5-8, and more preferably 6. The concentration of the buffer may be 10-200 mM, preferably 20 mM. Most preferably, the buffer is a sodium dihydrogen phosphate-disodium hydrogen phosphate buffer with a pH of 6 and a concentration of 20 mM.

In this preferred embodiment, the cross-linking reaction is performed in the presence of an oxidizing agent. The oxidizing agent is preferably a persulfate (e.g., ammonium persulfate), a divalent copper salt, a trivalent iron salt, a permanganate (e.g., potassium permanganate), or a periodate (e.g., sodium periodate), and more preferably ammonium persulfate. The mass concentration of the oxidizing agent in the solvent is 0.3-1.2 mg/mL, preferably 0.4-0.8 mg/mL, and more preferably 0.6 mg/mL.

In this preferred embodiment, the time of the cross-linking reaction is preferably overnight, for example, 12 hours.

In this preferred embodiment, the temperature of the cross-linking reaction may be room temperature, for example, 4-37° C., preferably 20-30° C.

Conventionally, the preparation method may further comprise post-treatment steps, such as centrifugation, washing (e.g., with PBS), and drying (e.g., freeze-drying).

On the basis of common knowledge in the art, each of the above preferred conditions can be arbitrarily combined to obtain each preferred embodiment of the present disclosure.

The reagents and raw materials used in the present disclosure are all commercially available.

The positive and progressive effects of the present disclosure lie in that the controlled-release preparation of the present disclosure has an ultra-high drug loading and a long release time, enabling long-term zero-order release of drugs. Moreover, the preparation method is simple and exhibits high encapsulation efficiency.

### BRIEF DESCRIPTION OF THE DRAWINGS

FIG. 1 is a schematic diagram illustrating the preparation of the controlled-release preparation of the present disclosure and its long-acting zero-order release.

FIG. 2 shows the scanning electron micrographs of recombinant human insulin crystals (a) and controlled-release preparation 1 (b), the transmission electron micrograph of a cryosection of controlled-release preparation 1 (c), and the fluorescence-labeled controlled-release preparation 1 (d) in Example 1.

FIG. 3 shows the scanning electron micrographs of insulin glargine crystals at different magnifications (a and b) and controlled-release preparation 4 at different magnifications (c and d) in Example 4.

FIG. 4 shows scanning electron micrographs of recombinant human insulin crystals (a) and controlled-release preparation 7 (b) in Example 7.

FIG. 5 shows scanning electron micrographs of recombinant human insulin crystals (a) and controlled-release preparation 8 (b) in Example 8.

FIG. 6 shows the cumulative release of recombinant human insulin in controlled-release preparations 1-3 (a), insulin glargine in controlled-release preparations 4-6 (b), and recombinant human insulin in controlled-release preparation 8 (c) in Effect Example 2.

FIG. 7 shows the blood glucose level curves of diabetic mice after subcutaneous injection of controlled-release preparations 1 and 4 in Effect Example 3.

### DETAILED DESCRIPTION OF THE PREFERRED EMBODIMENT

The present disclosure will be described in detail below by way of examples, but the scope of the present disclosure is not limited thereto. Experimental methods that do not indicate specific conditions in the following examples should be selected according to conventional methods and conditions, or according to product specifications.

### Example 1: Preparation of Controlled-Release Preparation 1 (Polydopamine Coating-Recombinant Human Insulin Crystal)

### 1. Preparation

Step 1: A disodium hydrogen phosphate-citrate buffer with a pH of 7 and a concentration of 182 mM was prepared;
Step 2: A dopamine solution with a concentration of 2 mg/mL and an ammonium persulfate solution with a concentration of 1.2 mg/mL were prepared using the above buffer;
Step 3: 10 mg of recombinant human insulin crystals were added to 2 mL of the dopamine solution and stirred uniformly with a magnetic stirrer;
Step 4: 2 mL of ammonium persulfate solution was added, and stirring was continued for 4 hours;
Step 5: The precipitate was centrifuged to the bottom of the centrifuge tube, washed with PBS three times, and freeze-dried.

### 2. Detection

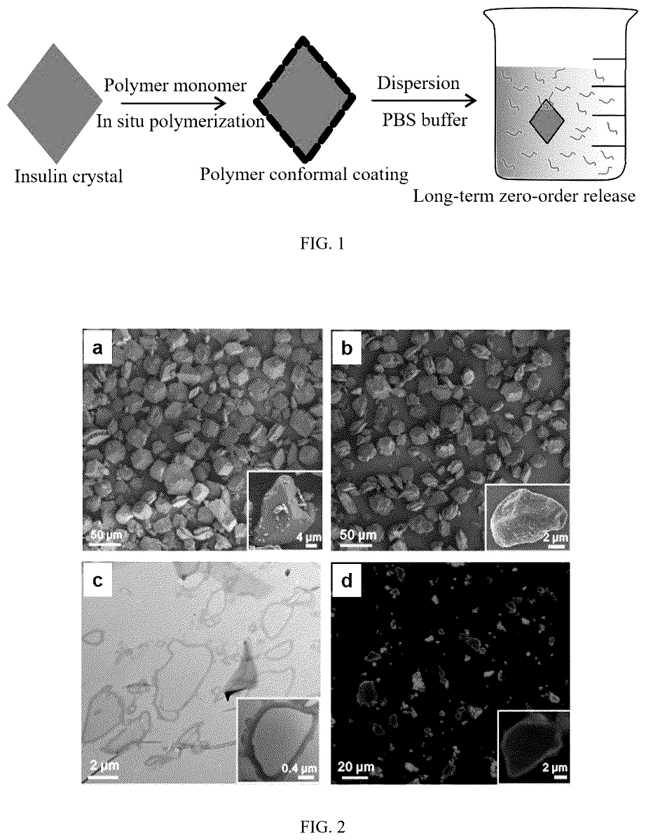

By comparing recombinant human insulin crystals (a of FIG. 2) and controlled-release preparation 1 (b of FIG. 2), it was observed from the scanning electron micrographs that the surface of the recombinant human insulin crystal became rough after in situ polymerization of dopamine.

The controlled-release preparation 1 was cryosectioned, and transmission electron microscopy revealed the formation of a uniform polymer film on the surface of the recombinant human insulin crystal (c of FIG. 2). The thickness of the polymer film was 161.54 nm, indicating that the drug crystal was encapsulated by the polymer film, confirming the successful preparation of controlled-release preparation 1.

### Example 2: Preparation of Controlled-Release Preparation 2 (Polydopamine Coating-Recombinant Human Insulin Crystal)

The preparation was carried out by the same method as in Example 1, except that the number of encapsulations was adjusted to 2, thereby forming controlled-release preparation 2.

### Example 3: Preparation of Controlled-Release Preparation 3 (Polydopamine Coating-Recombinant Human Insulin Crystal)

The preparation was carried out by the same method as in Example 1, except that the number of encapsulations was adjusted to 3, thereby forming controlled-release preparation 3.

### Example 4: Preparation of Controlled-Release Preparation 4 (Polydopamine Coating-Insulin Glargine Crystal)

The preparation was carried out by the same method as in Example 1, except that the recombinant human insulin crystal was replaced with the insulin glargine crystal.

By comparing b of FIG. 3 and d of FIG. 3, it was observed that the surface of the insulin glargine crystal became rough after in situ polymerization of dopamine, indicating that the insulin glargine crystal was encapsulated by a polydopamine film, thereby forming controlled-release preparation 4.

### Example 5: Preparation of Controlled-Release Preparation 5 (Polydopamine Coating-Insulin Glargine Crystal)

The preparation was carried out by the same method as in Example 4, except that the number of encapsulations was adjusted to 2, thereby forming controlled-release preparation 5.

### Example 6: Preparation of Controlled-Release Preparation 6 (Polydopamine Coating-Insulin Glargine Crystal)

The preparation was carried out by the same method as in Example 4, except that the number of encapsulations was adjusted to 3, thereby forming controlled-release preparation 6.

### Example 7: Controlled Release Preparation 7 (Poly-Levodopa-Recombinant Human Insulin Crystal)

The preparation was carried out by the same method as in Example 1, except that the dopamine solution was replaced with a levodopa solution.

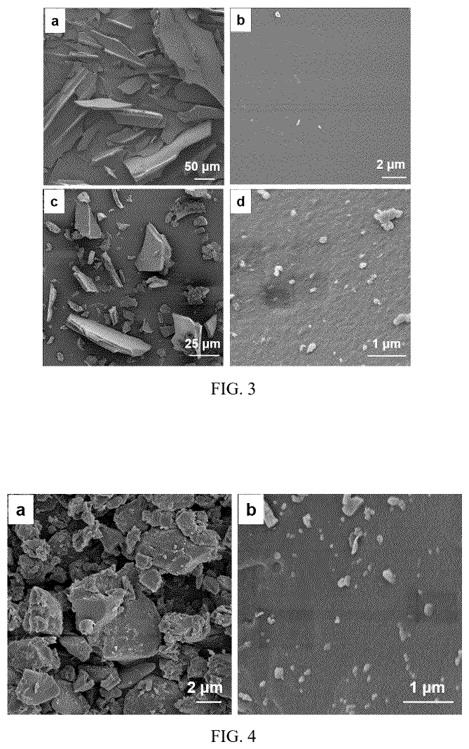

As can be seen from b of FIG. 4, the surface of the recombinant human insulin crystal became rough after in situ polymerization of levodopa, indicating that the recombinant human insulin crystal was successfully encapsulated by a poly-levodopa film, thereby forming controlled-release preparation 7.

### Example 8: Controlled Release Preparation 8 (Mussel Adhesive Protein-Recombinant Human Insulin Crystal)

### 1. Preparation

Step 1: 1 mg of mussel adhesive protein and 5 mg of recombinant human insulin crystals were dissolved in 1 mL of 20 mM, pH=6.0 sodium dihydrogen phosphate-disodium hydrogen phosphate buffer, and stirred for 2 hours;
Step 2: 0.6 mg of ammonium persulfate was added, and the reaction was stirred overnight;
Step 3: The precipitate was centrifuged to the bottom of the centrifuge tube, washed with PBS three times, and freeze-dried.

### 2. Detection

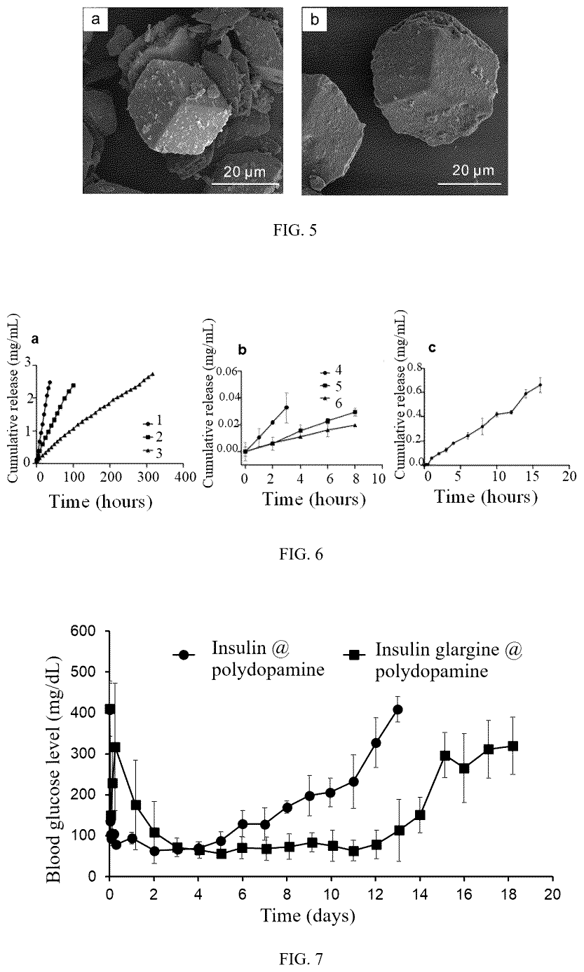

By comparing the scanning electron micrographs of recombinant human insulin crystals (a of FIG. 5) and controlled-release preparation 8 (b of FIG. 5), it was observed that the surface of the recombinant human insulin crystal became rough after cross-linking with mussel adhesive protein, thereby forming controlled-release preparation 8.

### Effect Example 1: Determination of Drug Loading and Encapsulation Efficiency

The drug loading and encapsulation efficiency of the above controlled-release preparations 1 to 6 and 8 were tested.

### Drug Loading Test Method:

A certain mass of the controlled-release preparation was weighed and dispersed in PBS buffer with pH=2 to dissolve the recombinant human insulin or insulin glargine in the controlled-release preparation. The concentration of recombinant human insulin or insulin glargine in the PBS buffer was determined by a microplate reader using the Coomassie Brilliant Blue method (detection wavelength: 595 nm), thereby calculating the mass of recombinant human insulin or insulin glargine in the controlled-release preparation. The drug loading was calculated using formula (1).

The calculation formula for drug loading was

$$Drug loading ( \% ) = \frac{\begin{matrix}Mass of recombinant human insulin or \\ insulin glargine in controlled - release preparation\end{matrix}}{Mass of controlled - release preparation} \times 100 \% \qquad ( 1 )$$

The calculation formula for encapsulation efficiency was

$$Encapsulation efficiency = \frac{Total mass - Unloaded mass}{Total mass} \times 100 \% \qquad ( 2 )$$

where the total mass was the mass of recombinant human insulin crystals or insulin glargine crystals added during the preparation process; the unloaded mass was the mass of recombinant human insulin or insulin glargine in the buffer after centrifugation, which was calculated based on the concentration of recombinant human insulin or insulin glargine measured by the Coomassie Brilliant Blue method (Formula (2)).

The drug loading and encapsulation efficiency data of controlled-release preparations 1 to 6 and 8 are shown in the following table:

| | Controlled-release preparation | | | | | | |
|---|---|---|---|---|---|---|---|
| | 1 | 2 | 3 | 4 | 5 | 6 | 8 |
| Drug loading | 92.91% | 77.13% | 65.80% | 91.31% | 87.34% | 60.03% | 82.70% |
| Encapsulation | 82.00% | 79.03% | 73.41% | 99.83% | 99.70% | 99.59% | 94.40% |
| efficiency | | | | | | | |

As can be seen from the above table, the controlled-release preparation of the present disclosure exhibited an ultra-high drug loading and encapsulation efficiency. The drug loading was decreased with the increase in the number of encapsulations, but all were greater than 60%. It was noteworthy that when encapsulated once, the drug loading was as high as 90% or more. The encapsulation efficiency of controlled-release preparations 1 to 3 decreased with the increase in the number of encapsulations, all being greater than 70%. The encapsulation efficiency of controlled-release preparations 4 to 6 was substantially maintained constant with the number of encapsulations, all being higher than 99%. Controlled-release preparation 8 exhibited both high drug loading and encapsulation efficiency, which were 82.7% and 94.4%, respectively.

### Effect Example 2: In Vitro Release Experiment

The controlled-release preparations 1-6 and 8 were subjected to in vitro release experiments, respectively, with the specific operations as follows:

The controlled-release preparations were dispersed in PBS buffer at a concentration of 5 mg/mL. The mixture was placed on a shaker and incubated at 37° C. At the set time point, 25 μL of liquid was taken, centrifuged to obtain the supernatant, and the concentration of recombinant human insulin or insulin glargine in PBS buffer was determined by the Coomassie Brilliant Blue method using a microplate reader (detection wavelength: 595 nm).

As shown in FIG. 6, after the drug crystal was encapsulated by the polymer film, drug release was effectively delayed, and zero-order release characteristics were exhibited. The release rate was slowed down as the thickness of the polymer coating increased. The controlled-release preparation 3 was able to maintain zero-order release behavior for 316 hours, with a cumulative release concentration of 2.74 mg/mL.

### Effect Example 3: In Vivo Release Experiment in Mice

Controlled-release preparation 1 and controlled-release preparation 4 were respectively used for the in vivo release experiment in mice, with the specific operations as follows:

6 mg of the above controlled-release preparation was weighed and dispersed in 0.5 mL of PBS buffer. After anesthetizing 5 type 1 diabetic mice, 0.1 mL of the controlled-release preparation in PBS buffer was injected subcutaneously into each diabetic mouse. Blood glucose levels at set time points were measured using a glucometer.

As shown in FIG. 7, after the controlled-release preparation was injected subcutaneously into diabetic mice, the in vivo release of insulin was significantly slowed. After the subcutaneous injection of the recombinant human insulin controlled-release preparation, the blood glucose level of diabetic mice was maintained at a normal level (approximately 200 mg/dL) for up to 10 days. After injection of the insulin glargine controlled-release preparation, the blood glucose level was maintained at a normal level (approximately 200 mg/dL) for up to 14 days. It can be seen that the controlled-release preparation of the present disclosure could significantly prolong the blood glucose regulation time of recombinant human insulin or insulin glargine.

Although specific embodiments of the present disclosure have been described above, it should be understood by those skilled in the art that these are merely illustrative examples, and various changes or modifications may be made to these embodiments without departing from the principles and essence of the present disclosure. Therefore, the scope of protection of the present disclosure is defined by the appended claims.

1. A controlled-release preparation, comprising a drug crystal and a polymer coating encapsulating the drug crystal.

2. The controlled-release preparation according to claim 1, wherein
the polymer is a dopa-modified polymer; or,
the drug is human insulin, an insulin analog, glucagon-like peptide-1, a hyperglycemic hormone, or an analgesic.

3. The controlled-release preparation according to claim 2, wherein the controlled-release preparation satisfies any one of the following conditions:
(1) the drug is human insulin or an insulin analog, and the polymer is polydopamine or poly-levodopa;
or (2) the drug is human insulin or an insulin analog, and the polymer is mussel adhesive protein.

4. The controlled-release preparation according to claim 1, wherein the controlled-release preparation consists of the drug crystal and the polymer coating encapsulating the drug crystal.

5. The controlled-release preparation according to claim 1, wherein the controlled-release preparation satisfies one or more of the following conditions:
(1) the particle size of the drug crystal is 10 nm-2 cm;
(2) the mass ratio of the drug crystal to the polymer coating is 1:(0.2-1.2);
(3) the thickness of the polymer coating is 50-1000 nm;
(4) the drug loading of the controlled-release preparation is 50%-99%; and
(5) the encapsulation efficiency of the controlled-release preparation is 60%-99.8%.

6. A preparation method for the controlled-release preparation according to claim 1, comprising the following step: subjecting a monomer to a polymerization reaction on the surface of the drug crystal to form the polymer coating.

7-8. (canceled)

9. The preparation method according to claim 6, wherein the polymerization reaction is carried out in the presence of a catalyst, and the catalyst is an oxidizing agent,
the oxidizing agent is a persulfate, a divalent copper salt, a trivalent iron salt, a permanganate, or a periodate; or,
the mass concentration of the oxidizing agent in the solvent is 0.3-1.2 mg/mL.

10. The preparation method according to claim 9, wherein
the time of the polymerization reaction is 2-12 hours; or,
the temperature of the polymerization reaction is room temperature.

11. A preparation method for the controlled-release preparation according to claim 1, comprising the following step: subjecting a polymer to a cross-linking reaction on the surface of the drug crystal to form the polymer coating.

12-13. (canceled)

14. The preparation method according to claim 11, wherein the cross-linking reaction is carried out in the presence of a catalyst, and the catalyst is an oxidizing agent,
the oxidizing agent is a persulfate, a divalent copper salt, a trivalent iron salt, a permanganate, or a periodate; or,
the mass concentration of the oxidizing agent in the solvent is 0.3-1.2 mg/mL.

15. The preparation method according to claim 11, wherein
the time of the cross-linking reaction is overnight; or,
the temperature of the cross-linking reaction is room temperature.

16. The controlled-release preparation according to claim 2, wherein the polymer is polydopamine, poly-levodopa, or mussel adhesive protein; or,
the drug is recombinant human insulin, insulin glargine, insulin detemir, insulin aspart, insulin lispro, insulin glulisine, low-protamine zinc recombinant human insulin, semaglutide, glucagon, tetracaine, or bupivacaine.

17. The controlled-release preparation according to claim 3, wherein the controlled-release preparation satisfies any one of the following conditions:
(1) the drug is recombinant human insulin or insulin glargine, and the polymer is polydopamine or poly-levodopa;
or (2) the drug is recombinant human insulin, and the polymer is mussel adhesive protein.

18. The controlled-release preparation according to claim 5, wherein the controlled-release preparation satisfies one or more of the following conditions:
(1) the particle size of the drug crystal is 0.1 μm-1000 μm;
(2) the mass ratio of the drug crystal to the polymer coating is 1:(0.2-0.8);
(3) the thickness of the polymer coating is 161.54 nm, 323.08 nm, and 484.62 nm;
(4) the drug loading of the controlled-release preparation is 60%-93%; and
(5) the encapsulation efficiency of the controlled-release preparation is 73.4%-99.8%.

19. The preparation method according to claim 6, wherein the operation of the polymerization reaction is as follows: mixing the monomer with the drug crystal in a solvent to carry out the polymerization reaction.

20. The preparation method according to claim 19, wherein the preparation method satisfies one or more of the following conditions:
(1) the drug is insulin or an insulin analog;
(2) the monomer is dopamine or levodopa;
(3) the mass ratio of the drug crystal to the monomer is 1:(0.2-1.2); and
(4) the mass concentration of the monomer in the solvent is 0.5-3 mg/mL.

21. The preparation method according to claim 19, wherein the solvent is a buffer, and the buffer satisfies one or more of the following conditions:
(1) the pH value of the buffer is 4-9;
(2) the buffer is a disodium hydrogen phosphate-citrate buffer, Tris buffer, or PBS buffer; and
(3) the concentration of the buffer is 10-200 mM.

22. The preparation method according to claim 11, wherein the operation of the cross-linking reaction is as follows: mixing the polymer with the drug crystal in a solvent to carry out the cross-linking reaction.

23. The preparation method according to claim 22, wherein the preparation method satisfies one or more of the following conditions:
(1) the drug is insulin or an insulin analog;
(2) the polymer is mussel adhesive protein;
(3) the mass ratio of the drug crystal to the polymer is 1:(0.2-1.2); and
(4) the mass concentration of the polymer in the solvent is 0.5-3 mg/mL.

24. The preparation method according to claim 22, wherein the solvent is a buffer, and the buffer satisfies one or more of the following conditions:
(1) the pH value of the buffer is 4-9;
(2) the buffer is a sodium dihydrogen phosphate-disodium hydrogen phosphate buffer; and
(3) the concentration of the buffer is 10-200 mM.
